(19) United States
(12) Patent Application Publication
(10) Pub. No.: US 2012/0045398 A1
(43) Pub. Date: Feb. 23, 2012

(54) Method For The Manufacture Of Nanoparticle Complexes And Triblock Polymer Ligands And Products Thereof

(76) Inventors: Elmar Pöselt, Hamburg (DE); Steffen Fischer, Bucholz (DE); Stephan Förster, Bayreuth (DE); Horst Weller, Hamburg (DE)

(21) Appl. No.: 13/224,818

(22) Filed: Sep. 2, 2011

Related U.S. Application Data
(63) Continuation-in-part of application No. PCT/EP2010/052567, filed on Mar. 3, 2010.

(30) Foreign Application Priority Data
Mar. 2, 2009 (GB) GB 0903448.9

(51) Int. Cl. A61K 49/18; C09K 11/54; C12N 9/96; C09K 11/02; B82Y 10/00; B82Y 5/00

(52) U.S. Cl. 424/9.322; 435/188; 424/9.3; 424/9.34; 424/9.35; 252/301.36; 977/774; 977/930; 977/834; 977/902; 977/773

(57) ABSTRACT

The present invention relates to the manufacture of triblock polymer ligands (30) and nanoparticle complexes (80). The nanoparticle complexes (80) comprise a capped nanoparticle (10) and the triblock polymer ligand (30). The triblock polymer ligand (30) consists of a binding polymer (40), a hydrophobic polymer (50) and a hydrophilic functionalisable polymer (60). The binding polymer (40) attaches to the capped nanoparticle (10).

### CROSS REFERENCE SECTION

This application claims priority under 35 U.S.C. §119 to Great Britain Application No. GB 0903448.9 filed Mar. 2, 2009 and under 35 U.S.C. §120 as a Continuation-in-Part to International Application No. PCT/EP2010/052567 filed Mar. 1, 2010 designating the U.S. and claiming priority to Great Britain Application No. GB 0903448.9. Both of these applications are incorporated herein in their entirety by reference.

### FIELD OF INVENTION

The present invention relates to the manufacture of triblock polymer ligands and their attachment to capped nanoparticles to produce functionalisable nanoparticle complexes.

### PRIOR ART

The manufacture of polymer ligands and their complexes with semiconducting nanoparticles is known in the art.

United States Patent Publication No. US 2004/0033345 which is owned by the University Rockefeller, N.Y. (USA) is titled, “Water soluble metal and semiconductor nanoparticle complexes.” The Rockefeller document discloses a water soluble complex which has an inner core of a metal or a semiconducting nanoparticle. The inner core is coated with a hydrophobic ligand and the nanoparticle is encapsulated in a micellar structure. In an aqueous medium the micellar structure comprises a hydrophilic shell and a hydrophobic core. The hydrophilic shell comprises a plurality of hydrophilic moieties. The hydrophobic core comprises a plurality of hydrophobic moieties, and each hydrophobic moiety comprises at least one chain and each chain comprises a minimum of 8 atoms, wherein the total number of atoms in all of the chains for each moiety comprises at least 24 atoms.

U.S. Pat. No. 6,319,426 B1, which is assigned to The Massachusetts Institute of Technology (MIT) is titled “Water-soluble fluorescent semiconductor nanocrystals”. The MIT patent discloses a water soluble semiconducting nanocrystal which is capable of light emission. The semiconducting nanocrystal has a semiconducting nanocrystal core which has a selected band gap energy, a shell layer which coats the semiconducting nanocrystal core and which is comprised of a semiconducting material having a band gap energy greater than that of the semiconducting nanocrystal, and an outer layer comprised of a molecule having at least one linking group for the attachment of the molecule to the coating shell layer. The water soluble semiconducting nanocrystal further comprises at least one hydrophilic group spaced apart from the linking group by a hydrophobic region, the hydrophobic region being sufficient to prevent electron charge transfer across the said hydrophobic region.

### BACKGROUND OF INVENTION

A range of applications in the field of medical science for semiconducting nanoparticles has become important during the last few years. This importance is due to the size-dependent, chemical and physical properties of high-quality II-VI semiconducting nanoparticles. Semiconducting nanoparticles are often referred to as quantum dots (QDs).

CdSe semiconducting nanoparticles have been investigated in fluorescent labelling due to their photo-luminescent efficiency. An advantage of the CdSe semiconducting nanoparticles is their size-dependent tuneable emission and the resistance of the CdSe nanoparticles to photo-bleaching. Due to the photo-luminescent efficiency of CdSe semiconducting nanoparticles they have the potential to be used as fluorescent markers in biological applications.

For use in biological applications the cytotoxicity of the CdSe semiconducting nanoparticles is a known problem. These problems arise from the release of Cd²⁺ ions from the CdSe semiconducting nanoparticles. In addition to the release of the Cd²⁺ ions the formation of free hydroxyl radicals can appear. The free hydroxyl radicals consequently damage the biological applications. The free hydroxyl radicals are generated by holes; this is by virtue of a valence band edge of 1.6 eV of the semiconducting CdSe nanoparticles.

A drawback associated with the cytotoxicity of the II-VI semiconducting nanoparticles has led to the manufacture of II-VI semiconducting nanoparticles with more than one shell, i.e. multi-shell semiconducting nanoparticles. In the multi-shell semiconducting nanoparticles an inorganic passivation layer on a nanoparticle surface ensures radiative electron and hole recombination. The inorganic passivation layer provides enhanced robustness against chemical degradation and photo-bleaching. Multi-shell II-VI semiconducting nanoparticles are known to exhibit a reduction in cytotoxicity when used in biological applications.

An example of the multi-shell semiconducting nanoparticle is where ZnS is used as the shell material for the semiconducting nanoparticle. ZnS has a band-gap energy of 3.8 eV and ZnS offers chemical stability and non-toxicity when used in biological applications. Furthermore, when two atomic layers of ZnS are on top of a semiconducting CdSe nanoparticle core; the two ZnS shells create a tunnel barrier with a band-gap energy of 4 eV and confine the wave function of the hole of an exciton inside the multi-shell semiconducting nanoparticle. However, this large mismatch of a band-gap energy (which is approximately 12%) between the semiconducting CdSe and semiconducting ZnS shell induces an interfacial strain between the core of the semiconducting nanoparticle and the shell of the semiconducting nanoparticle. The interfacial strain leads to a structural misfit and to dislocations of the core of the semiconducting nanoparticle with the shell of the semiconducting nanoparticle. This interfacial strain leads to a non-uniform surface of the multi-shell semiconducting nanoparticle and was reduced by the insertion of a CdS intermediate shell between the CdSe core and the outer ZnS shell.

The non-uniform surface of the multi-shell CdSe semiconducting nanoparticles results in a different co-ordination of the Cd and Se atoms within the core of the semiconducting nanoparticle. This different co-ordination is expressed by different binding energies of the passivation layer (e.g. a layer of ZnS) on the surface of the semiconducting nanoparticle core. This non-uniformity of the surface of the CdSe core affects the growth of the shell on the semiconducting nanoparticle core. It is known that ZnS shells grow preferably at anion rich sites of the core of the semiconducting nanoparticle of CdSe and consequently the shells of ZnS result in an incomplete coverage of the core of the CdSe semiconducting nanoparticle. This incomplete coverage of the core demonstrates a mechanism by which the binding of the passivation layer can influence the optical properties of the core of the multi-shell semiconducting nanoparticle.

Efforts have been made to exchange the semiconducting nanoparticle hydrophobic shells with hydrophilic ligands to form the passivation layer. The aim of the hydrophilic ligands is to achieve water solubility of the semiconducting nanoparticle. The hydrophobic nanoparticle shells of the semiconducting nanoparticle are known to kinetically stabilise the meta-stable semiconducting nanoparticles during the synthesis of the multi-shell semiconducting nanoparticle. Several studies and a number of ligands have been described in the literature to achieve efficient passivation layers on the surface of the semiconducting nanoparticles.

The paper by Algar et al. (see, W. R. Algar, J. Krull Ulrich, Journal a European Journal of Chemical Physics and Physical Chemistry, 2007, 8, 561.) describes changes in quantum yield of the semiconducting nanoparticles which are associated with changes in the radiative decay rates of the semiconducting nanoparticles. These changes in quantum yield of the semiconducting nanoparticles are directly associated with the nature of the ligand which is attached to the surface of the semiconducting nanoparticle, and to a smaller degree to the non-radiative decay rates of the semiconducting nanoparticles. The non-radiative decay rates of the semiconducting nanoparticles depend on the passivation layer and the subsequent exposure to a surrounding solvent matrix around the semiconducting nanoparticle. The non-radiative decay of the semiconducting nanoparticle shows that the degree of bathochromic shift observed is correlated to the degree and to the extent of ionisation of the ligand of the passivation layer of the semiconducting nanoparticle. The cause of the ligand chromism is directly correlated with the observed solvatochromism (see, C. A. Leatherdale, M. G. Bawendi, Physical Review B: Condensed Matter and Materials Physics 2001, 63, 165315/1.) in the CdSe semiconducting nanoparticles. The ligand chromism is due to the stronger dipolar interactions between the excited state of the passivation layer and the surrounding solvent matrix on the passivation layer on the semiconducting nanoparticle. Organic passivation layers are known to exhibit increasing red shifts of the respective emission maxima with increasing solvent polarity. Therefore in considering an inhomogeneous shell of a multi-shell semiconducting nanoparticle a dipolar or electrostatic interaction between the ligand and the solvent matrix and the semiconducting nanoparticle may be presumed. As a consequence the importance of the ligand attached to the semiconducting nanoparticle becomes relevant.

Regarding the cytotoxicity of the semiconducting nanoparticles when used in biological applications the use of a polymer ligand has become relevant. Such an example of the polymer ligand is poly (ethylene oxide) abbreviated as PEO. PEO is a hydrophilic polymer and is suitable for use in a number of biological applications. PEO is known to be inert and PEO is known to reduce non-specific accumulation in biological applications. The accumulation is ascribed to the cytotoxic effects in the biological applications and such cytotoxic effects are undesirable. A further polymer ligand is the hydrophobic ligand, poly (ε-caprolactone), abbreviated as PCL. PCL has been utilised in numerous biological applications by virtue of its biodegradability and biological compatibility. Furthermore a diblock polymer ligand of poly (ethylene oxide)-block-poly (ε-caprolactone), abbreviated as PEO-b-PCL has been shown to be an excellent drug carrier (see for example, M. A. R. Meier, S. N. H. Aerts, B. B. P. Staal, M. Rasa, U. S. Schubert, Macromolecular Rapid Communications 2005, 26, 1918 and F. Meng, C. Hiemstra, G. H. M. Engbers, J. Feijen, Macromolecules 2003, 36, 3004). In an aqueous medium, the diblock polymer ligand PEO-b-PCL forms a micellar structure upon complexation with the semiconducting nanoparticles. The formation of the micellar structure is facilitated by the hydrophobic PCL segment that stabilises the formation of the micellar structure. The hydrophobic PCL segment acts as a micro reservoir for the incorporation of lipophilic drugs. The hydrophilic PEO segment (which may feature functional groups) serves as an additional stabiliser to the micelle-like structure of the semiconducting nanoparticle complex.

The PCL polymer is hydrophobic and it is insoluble in an aqueous medium (i.e. biological applications). The nanoparticle core of the micellar nanoparticle complex which comprises the diblock polymer ligand PEO-b-PCL can be envisaged to be a quasi-solid. The quasi-solid facilitates a low critical micelle concentration. It is known that vesicle formation and biocompatibility with regards to drug delivery can be achieved when using the diblock polymer of a branched poly (ethylene imine)-poly-(ethylene oxide), abbreviated as PEO-PEI (see H. Petersen, P. M. Fechner, D. Fischer, T. Kissel, Macromolecules 2002, 35, 6867 and K. Kunath, A. von Harpe, H. Petersen, D. Fischer, K. Voigt, T. Kissel, U. Bickel, Pharmaceutical Research 2002, 19, 810).

The diblock polymer ligand PEO-PEI has previously been used for the phase transfer of CdSe/CdS core-shell semiconducting nanoparticles from an organic solvent to an aqueous medium.

The PEI polymer allows the bonding of the diblock polymer to the metal surface of the semiconducting nanoparticle.

The present disclosure teaches the manufacture of nanoparticle complexes with a hyper-branched PEI-PCL-PEO triblock polymer ligand. The triblock polymer ligand facilitates the phase transfer of the passivation layer of a plurality of different nanoparticles, including but not limited to iron oxide nanoparticles, gold nanoparticles, CdSe nanoparticles, CdSe/CdS core/shell nanoparticles, CdSe/CdS/ZnS core/shell/shell nanoparticles from an organic solvent to an aqueous medium. The nanoparticle complex possesses a homogenous inorganic passivation layer in the aqueous medium, whilst the nanoparticle complex 80 maintains high rigidity and chemical stability.

Furthermore, the triblock polymer ligand bears terminal functional groups. The terminal functional groups of the triblock polymer ligand can be utilised for further coupling reactions. The method for the manufacture of the nanoparticle complex and the triblock polymer ligand provides a simple and versatile strategy to achieve a water-soluble semiconducting nanoparticle.

### SUMMARY OF INVENTION

The present invention discloses a method for the manufacture of a triblock polymer ligand. The triblock polymer ligand is used to form a nanoparticle complex with a capped nanoparticle. The triblock polymer ligand provides the nanoparticle complex with a homogenous organic passivation layer on the surface of the capped nanoparticle. The resulting nanoparticle complex has enhanced luminescent stability when used in biological applications.

The triblock polymer ligand is functionalisable and can be further functionalised for use in a wide range of specific applications depending upon the required use.

The nanoparticle complexes are soluble in aqueous mediums and can be used in biological applications. Biological applications include MRT, colorimetric sensing, optical imaging, electrochemical sensing, attachment to magnetic particles (allowing movement using magnetic field), biomedical imaging and attachment to further molecules and biomolecules like enzymes, antibodies, and carbohydrates. Hydrophobic molecules can be encapsulated inside the micelle in the process of self-assembly, concerning for example drug delivery vehicles. Clustering of different types of nanoparticles in one micelle and the attachment of further markers (radioactive, fluorescence) is possible.

The triblock polymer ligands can be used to form complexes around a single nanoparticle or a cluster of nanoparticles. Clustering of iron oxide nanoparticles may result in complexes with altered magnetic properties, which make them suitable as MRT contrast agents.

The triblock polymer ligand can be used to form nanoparticle complexes with multi-shell nanoparticles or nanoparticles which are not semiconducting nanoparticles.

The capped nanoparticles are preferably semiconducting nanoparticles.

In a further aspect of the present invention the triblock polymer ligand can be used to solubilise any hydrophobic nanoparticle in an aqueous medium.

### DESCRIPTION OF FIGURES

FIG. 1 show a schematic of a nanoparticle complex 80 manufactured in accordance with the present invention.

FIG. 2 shows a scheme for the manufacture of diblock polymer 20.

FIG. 3 shows the ¹H NMR of a hydrophilic functionalisable polymer 60.

FIG. 4 shows the ¹H NMR of three different triblock polymer ligands 30, with different block sizes (PEO 1100 g/mol to 4500 g/mol) and thermal groups according to different aspects of the invention.

FIG. 5 shows the ¹H NMR of core/shell/shell capped nanoparticles CdSe/CdS/ZnS during the addition of the ligand M-PEO₁₁₀₀-NB4.

FIG. 6 shows a bonding scheme of ligand to a capped nanoparticle (upper schematic) and the bonding of the passivation layer to the capped nanoparticle (lower schematic).

FIG. 7 shows CdSe/CdS/ZnS multi-shell capped semiconducting nanoparticle complex with the ligand tC-PEO-PCL-PEI (CP) in water. a) absorbance spectra and b) emission spectra.

FIG. 8 shows a graph in which the emission of CdSe/CdS/ZnS multi-shell capped semiconducting nanoparticle complex with the ligand tC-PEO-PCL-PEI (DPA) is plotted against concentration in water in the progress of dilution.

FIG. 9 shows: CdSe/CdS/ZnS multi-shell capped semiconducting nanoparticle complex with the triblock polymer ligand DP-PEO-PCL-PEI (DPA) in water. a) DLS intensity b) DLS volume c) DLS number.

FIG. 10 shows, a) TEM image b) confocal microscope image of CdSe/CdS/ZnS capped nanoparticle complex with the triblock polymer ligand DP-PEO-PCL-PEI (DPA) in water.

FIG. 11 shows functionalisable possibilities of the nanoparticle complex 80.

FIG. 12 shows the nanoparticle complex 80 in one of the observed structures of the present invention.

FIG. 13 shows TEM images of CdSe/CdS/ZnS coated by M-PEO-PCL-PEI triblock polymer ligand 30.

FIG. 14 shows results of buffer tests of CdSe/CdS/ZnS nanoparticles, with different coatings.

FIG. 15 shows a TEM image of super paramagnetic iron oxide particles incubated with the PEO-PCL-PEI triblock copolymer in THF after transfer to a TEM-grid.

FIG. 16 shows the FTIR-spectrum proving the hydrogen bonding interactions of the hydrophobic polymer (50) (PCL) with the binding polymer (40) PEI.

FIG. 17 shows the dependence of the r2 relaxivity on the hydrodynamic diameter of the encapsulated iron oxide nanoparticles inside a triblock copolymer (30) micelle.

FIG. 18 Comparison of fluorescence intensity for nanoparticle complexes in aqueous solution made from an example of a triblock copolymer 30 (PEO-PCL-PEI) and a diblock copolymer PEO-PEI where no hydrophobic block 50 is present respectively.

FIG. 19 WTS assay for assessing cytotoxicity of the pure triblock copolymer PEO-PCL-PEI

FIG. 20 WTS assay for assessing cytotoxicity of triblock copolymer (PEO-PCL-PEI) encapsulated super paramagnetic iron oxide nanoparticles complexes.

FIG. 21: TEM images of FeOₓ (diameter ˜22 nm) and NiPt (diameter ˜8 nm) nanoparticles coated with ligand [3,3-Dieethoxy-propanyl-PEO₁₆₀₀-PCL₄₀₀]₂₋₃-PEI₇₀₀ after evaporation of the prior solvent water

### DETAILED DESCRIPTION

For a complete understanding of the present invention and the advantages thereof, reference is made to the following detailed description in conjunction with the accompanying Figures.

It should be appreciated that the various aspects of the present invention discussed herein are merely illustrative of specific ways to make and use the invention and do not limit the scope of invention when taken into consideration with the claims and the following detailed description and the accompanying Figures.

It should be realised that features from one aspect of the invention can be combined with features from other aspects of the invention. The use of the term “a”, “an” and “the” as used throughout the description includes plural references unless clearly indicated otherwise.

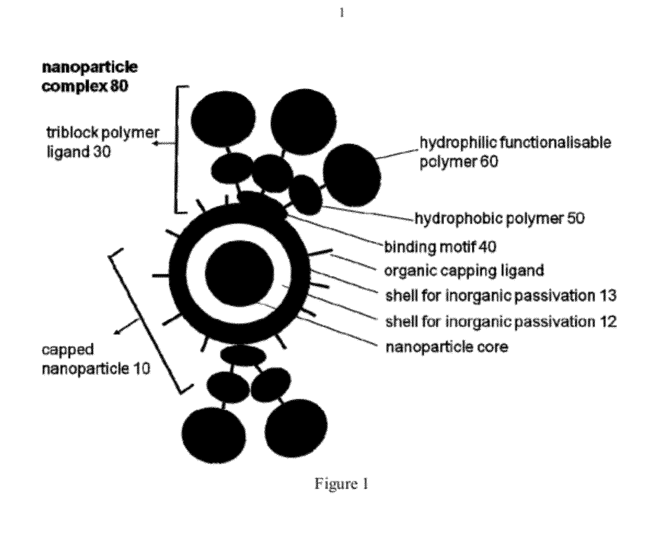

FIG. 1 shows an example of a nanoparticle complex manufactured in accordance with this invention. Capped nanoparticles 10 are preferably semiconductor nanoparticles with optical and electronic properties. The capped nanoparticles 10 have a core 11 of one material and may be surrounded by a shell 12 of another material to give a core-shell capped nanoparticle 10. The core 11 may be surrounded by two shells to give a core-shell-shell nanoparticle. The two shells serve as an inorganic passivation layer. The material of the core 11 and the material of shell 12 can be of the same or of different material. The material of the core 11 and the material of shell 12 may be a semiconducting material. The semiconducting material of the shell preferably has a band-gap energy that is greater than the band-gap energy of the semiconducting material of the core 11. The capped nanoparticles 10 preferably have an organic layer of TOP/TOPO on the surface of the semiconducting nanoparticle.

Examples of the capped nanoparticles 10 include but are not limited to the following nanoparticles species:
alkylamine capped nanoparticles, e.g. dodecylamine capped CdSe nanoparticles.
fatty acid capped nanoparticles, e.g. oleic acid capped CdSe nanoparticles or oleic acid capped iron oxide nanoparticles.
trialkylphosphine/trialkylphosphineoxide capped nanoparticles, e.g. trioctyl phosphine/trioctyl phosphine oxide (TOP/TOPO) capped CdSe nanoparticles.
capped nanoparticles prepared in an unpolar hydrocarbon solvent, like alkanes, e.g. dodecane, or unsaturated hydrocarbons e.g. squalene.
rare earth doped nanoparticles, e.g. NaYF₄, or GdPO₄,
elements of the 2A/B group and 6A group of the periodic table, e.g. CdS, CdSe, ZnO, ZnS or CdTe.
elements of the 4A group and 6A group of the periodic table, e.g. PbS.
elements of the 3A group and 5A group of the periodic table, e.g. InP or InAs.
noble metal, e.g. gold, silver or platinum.
alloy, e.g. NiPt, FePt.
metal oxide, e.g. Fe₂O₃ or Fe₃O₄

Examples of the capped nanoparticles (10) which are core/shell nanoparticles include but are not limited to:
a core nanoparticle and a shell comprised of elements of the 2A group and 6A group of the periodic table, e.g CdTe/CdS.
core nanoparticle and a shell comprised of elements of the 2A/B group and 6A group of the periodic table, e.g PbS/CdS.
a core nanoparticle and a shell comprised of elements of the 2A/B group and 6A group of the periodic table, e.g InAs/CdSe or InP/ZnS.

Examples of the core/shell/shell nanoparticles comprised of a core nanoparticles include shells comprised of elements of the 2A/B group and 6A group of the periodic table, e.g CdSe/CdS/ZnS, CdSe/ZnSe/ZnS, CdSe/ZnCdS alloy/ZnS

The triblock polymer ligand 30 comprises a binding motif 40, a hydrophobic polymer 50 and a functionalisable hydrophilic polymer 60. The term binding motif 40 is also used to describe binding polymers 40, as seen in different examples of the present invention.

The binding polymer 40 of the triblock polymer ligand 30 is a hyper branched polymer which has a number of nucleophilic groups (such as one or more amines, phosphates or thiols). The nucleophilic groups are generally Lewis bases.

The nucleophilic groups allow the triblock polymer 30 to bond to the shell 12 of the capped nanoparticle 10 to form a nanoparticle complex 80.

The binding polymer 40 should exhibit characteristics for complexation of the triblock polymer ligand 30 to the capped nanoparticle 10. Such characteristics are relevant with regards to the nucleophilic moieties which may be present in the binding polymer 40. These characteristics are relevant when the nanoparticle complex 80 is used in biological applications. These characteristics are:

1. The ligand 30 needs to be strongly bonded to the capped nanoparticle 10 surface. This strong bonding ensures the preservation of physical properties of the capped nanoparticle 10 as well as the continuance of coupled molecules like proteins and leads to the reduction of potential cytotoxicity of the nanoparticle core 11.

2. The polymer 60 should offer a functional moiety towards an aqueous medium. The functional moiety should not lead to a crosslink between different capped nanoparticles 10 and should offer a functional group for further modification.

3. The functionalisable hydrophilic polymer 60 should allow for the mild coupling reactions for proteins in essential buffer solutions.

4. The hydrophobic polymer block 50 has to form a hydrophobic shell around the capped nanoparticles which shields the inner part of the micelle from the aqueous environment.

5. The binding polymer 40 and the hydrophobic polymer 50 have the ability to interact via hydrogen bonds.

To combine all of these characteristics, the triblock polymer ligand 30 is manufactured and comprises three polymer segments.

The functionalisable hydrophilic polymer 60 aids the solubility of the nanoparticle complex 80 in an aqueous medium. Non-limiting examples of hydrophilic polymer 60 are polyalcohols, polysugars like dextranes, polyoxazolines, hydrophilic (poly-)peptides. In an example of the present invention the functionalised hydrophilic polymer 60 is a poly (ethylene oxide) polymer.

The functionalisable hydrophilic polymer 60 has terminal functional groups that allow the triblock polymer ligand 30 to be further functionalised before or after manufacture of the nanoparticle complex 80.

The hydrophobic polymer 50 can be any hydrophobic polymer, which is able to act as a hydrogen bond donor or acceptor. Non-limiting examples for suitable hydrophobic polymer 50 segments are:

Polyester like Polyethylene terephthalate (PET), Polyethylene naphthalate (PEN), Polybutylene terephthalate (PBT), Polytrimethylene terephthalate (PTT), Polyglycolide or Polyglycolic acid (PGA), Polylactic acid (PLA), Polycaprolactone (PCL), Polyethylene adipate (PEA), Polyhydroxyalkanoate (PHA), Vectran, Polycyclohexylenedimethylene terephthalate (PCT); Polycarbonates like allyl diglycol carbonate (ADC), hydrophobic peptides like transmembrane helices from transmembrane proteins; Polyethers; Polyetherimides; Polyesteramides; Polyamides like Polycaprolactam, Acrylate Polymers like Poly(methyl methacrylate) (PMMA); Polyvinyl acetate (PVAc)Polymethacrylmethylimid (PMMI); Polyamide-imides; Polyether ether ketone (PEEK); Polyimide like Polybismaleinimid (PBMI); Polyureas; Polyvinylpyrrolidon (PVP) Polyhydantoine

The hydrophobic polymer 50, in an example of the present invention, is a poly (ε-caprolactone) polymer.

The binding polymer 40 is a linear or branched polymer baring multiple functional groups. The binding polymer needs to fulfil two requirements. First it needs to have nucleophilic groups with an affinity for the inorganic nanoparticle material and secondly it has to act as a hydrogen bond donor or acceptor to interact with the corresponding donor or acceptor groups in the hydrophobic block 50. The binding to the nanoparticle surface and the interactions with the hydropbobic block take place at the same time.

The binding polymer 40, in one example of the present invention, is a hyper-branched polymer. In an example of the present invention the binding polymer 40 is a poly (ethylene imine) polymer (PEI polymer). The PEI polymer possesses an average of 6 to 8 primary and/or secondary amines. The binding polymer 40 has a length which is approximately less than one third of that of the hydrophilic functionalisable polymer 60.

The triblock polymer ligand 30 was manufactured initially by the manufacture of a diblock polymer 20. The diblock polymer 20 comprises the hydrophilic functionalised polymer 60 and the hydrophobic polymer 50. Each triblock polymer ligand 30 bears a mean average of 2 or 3 diblock polymers 20 coupled to one binding polymer 40 and the total mass of the triblock polymer ligand 30 is approximately 6000 g/mol.

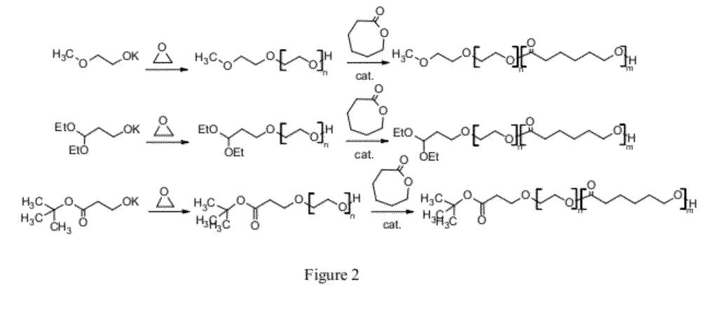

The manufacture of the diblock polymer 20 proceeds in accordance with the schematic shown in FIG. 2. The diblock polymer 20 comprises the hydrophobic polymer 50 and the functionalisable hydrophilic polymer 60. The functionality of the functionalised hydrophilic polymer 60 stems from its initial synthesis whereby the functionalisable hydrophilic polymer 60 bears functional terminal groups.

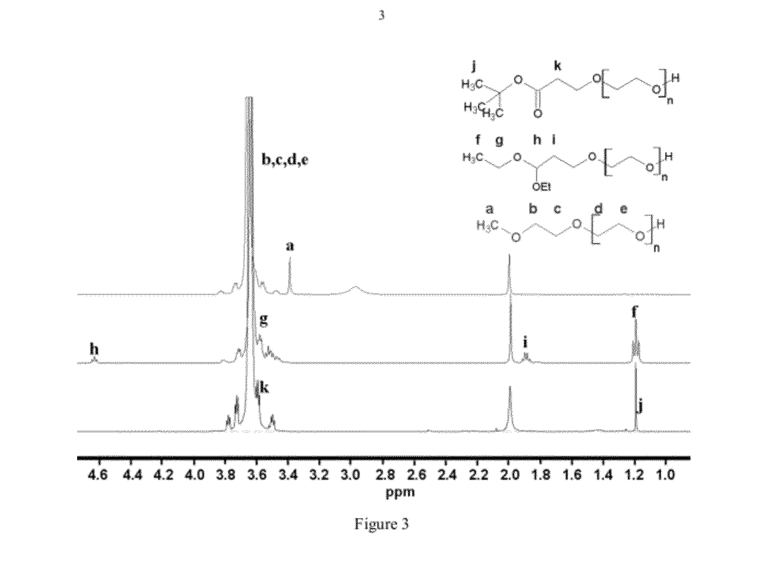

The manufacture of the diblock polymer 20 is achieved by a ring opening polymerisation reaction. The hydrophilic functionalisable polymer 60 is manufactured by reacting potassium salts of 2-methoxyethanol (M-) (upper schematic of FIG. 2), 3,3-diethoxy-propanol (DP-) (middle schematic of FIG. 2) and tert-butyl-3-3-hydroxypropinoate (tC-) (lower schematic of FIG. 2) with ethylene oxide. Thus the potassium salt of DP-, tC- and M-, provides the terminal functional group moiety of the hydrophilic functionalisable polymer 60. The hydrophilic moiety is provided by the ethylene oxide of the hydrophilic functionalisable polymer 60. The amount of ethylene oxide used in the anionic ring opening polymerisation reaction is proportional to the length of the hydrophilic functionalisable polymer 60. FIG. 3 shows the ¹H NMR of the three examples of the hydrophilic functionalisable polymers 60 according to the present invention. The upper schematic of FIG. 3 shows ¹H NMR of M-PEO, the middle schematic shows the ¹H NMR of DP-PEO and the lower schematic shows the ¹H NMR of tC-PEO.

The next stage of the manufacture of the triblock polymer ligand 30 is the manufacture of the diblock polymer 20 from the functionalisable hydrophilic polymer 60. With reference to FIG. 2, this is achieved by polymerisation of ε-caprolactone, the ε-caprolactone adds to the hydrophilic functionalisable polymer 60. The ε-caprolactone in this example of the present invention provides the hydrophobic feature to the hydrophobic polymer 50.

Three examples of the diblock polymer 20 are shown in FIG. 2. M-PEO-PCL (upper right schematic of FIG. 2), DP-PEO-PCL (middle right schematic of FIG. 2) and tC-PEO-PCL (lower right schematic of FIG. 2). The amount of ε-caprolactone which is added to the hydrophilic functionalisable polymer 60 is proportional to the length of the hydrophobic polymer 50 segment. In the three examples of the present invention, the diblock polymer 20 is soluble in polar solvents such as dichloromethane.

The hydrophobic polymer 50 is a hydrophobic substance. Therefore a characteristic of the diblock polymer 20 is that the morphology and the solubility of the diblock polymer 20 in water depend upon the size of the hydrophobic polymer 50.

The next step for the manufacture of the triblock polymer ligand 30 is the coupling of the binding polymer 40 to the diblock polymer 20. In an example of the present invention the binding polymer 40 has hyper branched nucleophilic groups and the binding polymer 40 in an example of the present invention is a poly (ethylene imine) polymer, abbreviated as PEI. The binding polymer 40 has nucleophilic moieties that allow it to bind the metal surface of the capped nanoparticle 10.

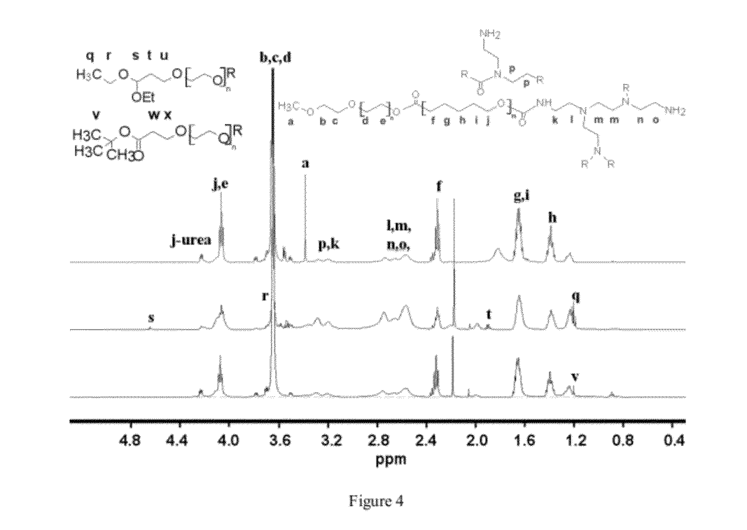

The coupling of the diblock polymer 20 to the binding polymer 40 proceeds by the use of a coupling agent. An example of the coupling agent is as 1,1-carbodiiimadiazol (CDI). The coupling agent is used in a 9 to 15 fold excess in relation to the diblock polymer 20 and the binding polymer 40. The diblock polymer 20 and the binding polymer 40 are present in a 1-3:1 ratio. FIG. 4 displays the ¹H NMR of the triblock polymer ligand 30 according to three examples of the present invention, M-PEO-PCL-PEI (upper schematic of FIG. 4), DP-PEO-PCL-PEI (middle schematic of FIG. 4) and tC-PEO-PCL-PEI (lower schematic of FIG. 4).

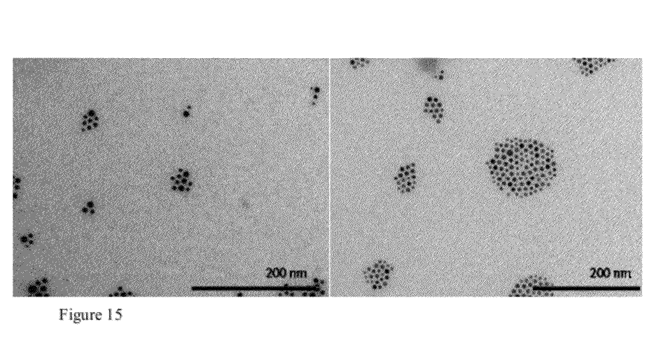

The manufacture of the nanoparticle complex 80 is achieved by forming a mixture 65 of the capped nanoparticle 10 and the triblock polymer ligand 30 in a liquid 70. The mixture 65 is made up of between 0.5 nmol and 10 nmol of the capped nanoparticle 10 and a 1 to 5 hundred molar excess of the triblock polymer ligand 30. The capped nanoparticle 10 and the triblock polymer ligand 30 present in the mixture 65 are incubated in the liquid 70. The mixture 65 and the liquid 70 are a solution. The liquid 70 in an example of the present invention is anhydrous tetrahydrofuran (THF). In this stage the particles do already form clusters in solution when the triblock copolymer ligand 30 is added in a ratio preventing that each single capped nanoparticle 10 can be coated by the ligand. FIG. 15 shows a TEM image of the nanoparticle-triblock copolymer mixture from THF. Distinct assemblies of particles are already visible showing that the clusters do not only form in the presence of water.

The final nanoparticle complex 80 is obtained by the addition of distilled, deionised water to the mixture 65 after the mixture 65 has been incubated to yield the nanoparticle complex 80.

The nanoparticle complex 80 can be formed without an incubation period, i.e. directly in the mixture 65. In the example of the invention in which THF was used as the liquid 70, the incubation period was approximately five minutes. THF is preferred as the liquid 70 since THF is hydroscopic. The hydroscopic nature of THF facilitates the removal of the THF since THF is miscible with water and has a low boiling point which facilitates its removal after the nanoparticle complex 80 has been manufactured. However in other examples of the present invention chloroform has been used as the liquid 70. The chloroform remains present in the micellar structure of the nanoparticle complex 80. It has been observed that in the example in which chloroform is used as a liquid 70 a higher quantum yield of the nanoparticle complex 80 was achieved.

The ligand addition or partial exchange can be achieved by different methods.

The triblock polymer ligand 30 can be added to the capped nanoparticle 10 or vice versa. The mixed solution of the triblock polymer ligand 30 together with the capped nanoparticle 10 in a water miscible organic solvent (e.g. THF) is added into water or vice versa.

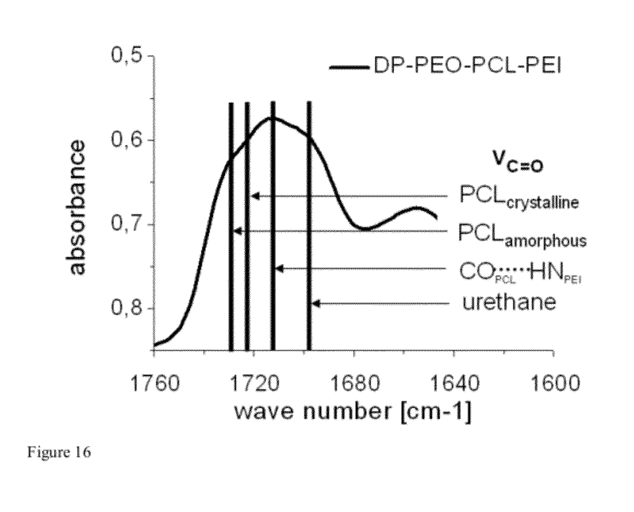

The triblock polymer ligand 30 facilitates effective complexation to the capped nanoparticle 10 to produce the nanoparticle complex 80. Effective complexation is achieved, not only by the multi-attachment of the linear or hyper branched nucleophilic moieties of the binding polymer 40, but also by the hydrophobic interactions of the hydrophobic polymer 50 with other hydrophobic polymer-block 50 segments of neighbouring triblock polymer ligand 30 and with the octyl chains of the TOP/TOPO passivation layer present on the surface of the capped nanoparticle 10. It also observed that hydrogen bonding interactions between ester groups in the hydrophobic polymer 50 (when the hydrophobic polymer 50 is PCL) with the nucleophilic amine groups of the binding polymer 40 (when the binding polymer 40 is PEI) are present. The presence of hydrogen bonds is shown in FIG. 16 below where a shift in peak position of the resonance maximum of the C═O vibration is observed in FTIR-spectrum relative to the free ester bond. This shift in resonance frequency upon hydrogen bond formation has been reported by (X. Shuai, T. Merdan, F. Unger, M. Wittmar, T. Kissel, Macromolecules 2003, 36, 5751.) It can be seen from FIG. 16 that unbound amorphous PCL and hydrogen bonded PCL are coexistent in the triblock-copolymer layer.

Tight packing of the TOP/TOPO passivation layer present on the capped nanoparticle 10 with the hydrophobic polymer 50 is observed. The tight packing protects the core 11 of the nanoparticle 10 from outer milieu of the micellar structure of the nanoparticle complex 80. Therefore, the nucleophilic amines of the binding polymer 40 are shielded from the outer milieu of the micellar structure of the nanoparticle complex 80. Also the unbound triblock polymer ligands 30 in water exhibits a morphology in water which is in the expected size range of empty micelles.

Due to the nucleophilic character of amines present in the binding polymer 40 shielding of the unbound ligand prevents the amines of the binding polymer 40 from unfavourable side reactions during the reaction nanoparticle complex 80 with molecules like proteins. Since the amines are encapsulated inside the micellar structure of the nanoparticle complex 80, the amines cannot interfere with the binding of the triblock polymer ligand 30 with the nanoparticle 10. The bond strength to the nanoparticle surface can be advanced easily by amplifying a part of the amines with carbon disulfide (CS₂). The obtained bidentate chelating dithiocarbamate groups (NH—C(S)S—) exhibit a significant enhanced stability against photo-oxidation, which is not the case for thiol ligands. The photooxidation is accompanied by the formation of disulfides, which leads to the precipitation of the nanoparticle.

Ligand addition of the PEO-N3 ligand to the multi-shell capped nanoparticle 10 of CdSe/CdS/ZnS was followed by ¹H-NMR spectroscopy in order to follow the ¹H resonance changes of the organic TOP/TOPO layer.

To effectively observe ¹H-NMR spectroscopy and nuclear magnetic resonance changes during the addition of the PEO-N3 ligand, any unbound TOP/TOPO was removed from the capped nanoparticle 10. Therefore the capped nanoparticle 10 was purified from excess amounts of unbound TOP/TOPO to the capped nanoparticle 10 by precipitation of the capped nanoparticle 10 with methanol. The remaining bound TOP/TOPO passivation layer on the capped nanoparticle 10 is necessary to facilitate the solubility of the capped nanoparticle 10 in anhydrous CDCl₃ during the ¹H-NMR studies.

Furthermore a new ligand M-PEO₁₁₀₀-N4 was prepared to avoid signal overlap for measuring the progress of the addition of the triblock polymer 30 to the capped nanoparticle 10. The new ligand has a M-PEO₁₁₀₀ polymer segment with a mass of 1100 g/mol and comprises the binding polymer 40 of tris (2-aminoethyl amine) is abbreviated as -N4.

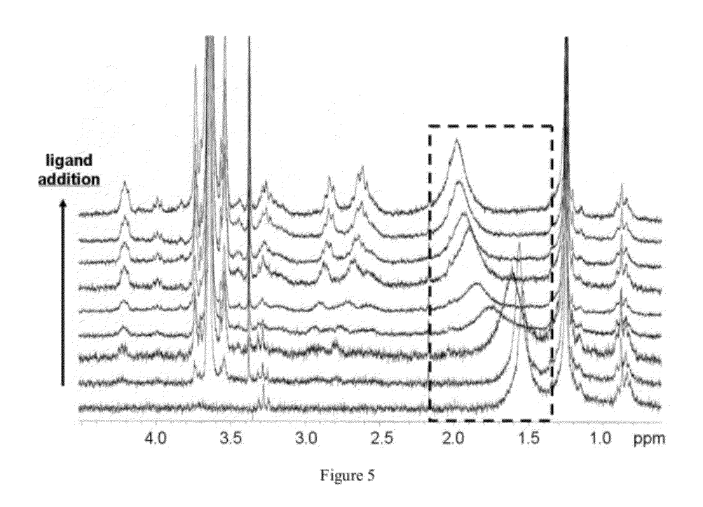

¹H-NMR measurements were performed with the multi-shell capped nanoparticle 10 of CdSe/CdS/ZnS (see FIG. 5 of the ¹H-NMR spectrum) and after each addition of a 25 fold excess of the M-PEO₁₁₀₀-N4 ligand with an incubation time of 20 minutes. The signal broadening and partial overlap of the first three methylene groups close to the phosphorus atom TOPO at 1.5 ppm arises. This is due to the steric hindrance concerning relaxation of the first three methylene protons. This observed relaxation is in agreement with the findings of L. Shen, R. Soong, M. Wang, A. Lee, C. Wu, G. D. Scholes, P. M. Macdonald, M. A. Winnik, Journal of Physical Chemistry B 2008, 112, 1626.

During the analysis of the ligand addition with the M-PEO₁₁₀₀-N4, the TOP/TOPO ¹H signal shifts from 1.5 ppm to 1.9 ppm (FIG. 5, boxed area). The ¹H shift is accompanied with a broadening of the ¹H signal. Furthermore since the separate ¹H signals of the methylene ¹H adjacent to the phosphorus atom do not emerge. This non emergence implies that the TOP/TOPO molecules of the passivation layer do not detach from the surface of the capped nanoparticle 10 during the manufacture of the nanoparticle complex. The broadening of the ¹H signal in regards to the impeded relaxation is due to the steric requirements of the capped nanoparticle 10 support the fact that the TOP/TOPO molecules of the passivation layer do not detach from the surface of the capped nanoparticle 10.

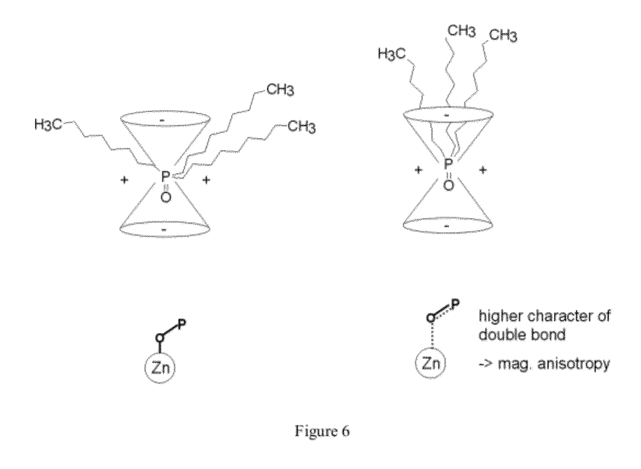

The steric oppression of the ¹H signals of the TOP/TOPO passivation layer could crowd the alkyl chains in the reduced shielded area of the magnetic anisotropic phosphine oxide of the TOP/TOPO (as illustrated in FIG. 6, upper schematic), whereby nearby ¹H show a deep field chemical shift, i.e. higher δ-values. This effect of a deep field chemical shift can be supported in changes in the bonding behaviour of the TOP/TOPO passivation layer of the capped nanoparticle 10 during the manufacture of the nanoparticle complex 80. The change in bonding is dative bonding. The dative binding across oxygen of the P═O group could therefore be elongated, increasing the double bond character of P═O bond and consequently increase the anisotropy of the P═O bond (as illustrated in FIG. 6, lower schematic).

The chemical shift observations demonstrate the close proximity of the TOP/TOPO passivation layer of the capped nanoparticle 10 with the M-PEO₁₁₀₀-N4 ligand. This close proximity arrangement increases the van der Waals forces between poly-methylene groups of the TOP/TOPO passivation layer and the nucleophilic -N4 of the ligand. The increase in the van der Waals forces is in agreement with the results of Young et al. (A. G. Young, N. Al-Salim, D. P. Green, A. J. McQuillan, Langmuir, American Chemical Society—awaiting publication).

Transferring the inferences with regards to the proximity of the M-PEO₁₁₀₀-N4 ligand to the surface of the capped nanoparticle 10 of the nanoparticle complex, it is envisaged that the triblock polymer ligand 30 of PEO-PCL-PEI is attached to the capped nanoparticle 10, where no excess TOP/TOPO has been removed. An extremely close hydrophobic polymeric barrier from the hydrophobic polymer 50 around the surface of the capped nanoparticle 10 is observed. On the basis of this observation the nanoparticle complex should offer a tight ligand packing, which efficiently shields the nanoparticle from the outside milieu, that leads to enhanced stability of the nanoparticle complex 80 when used in biological applications.

A further advantage of the triblock polymer ligand 30 PEO-PCL-PEI is that the triblock polymer ligand 30 facilitates the avoidance of ligand exchange processes. The avoidance of ligand exchange processes implies the rugged precipitation of the capped nanoparticles 10 to remove the TOP/TOPO layer. The TOP/TOPO layer is not displaced as there is mainly a ligand addition process.

Semiconducting CdSe capped nanoparticles 10 are known to exhibit solvatochromism (see for example, C. A. Leatherdale, M. G. Bawendi, Physical Review B: Condensed Matter and Materials Physics 2001, 63, 165315/1.). Solvatochromism is where local dielectric properties and interactions of the nanoparticle passivation layer with a surrounding matrix influence the optical transition energy of the semiconducting capped nanoparticles 10.

In the present invention it is observed that the solvatochromism is minimised by the micelle-like structure of the nanoparticle complex 80. The micellar structure provides a chemical environment similar to that of the initial organic solvent of the surrounding solvent matrix. The inert PEO hydrophilic functionalisable polymer 60 ensures rigidity of the micellar structure against biological degradation. This rigidity is significant since the hydrophobic polymer 50 PCL is known to be biodegradable. The water does not come into contact with the capped nanoparticle 10.

FIG. 1 shows the bonding polymer 40 attached to the capped nanoparticle 10. The bonding polymer 40 is attached to the capped nanoparticle 10. The hydrophobic polymer 50 is attached to the bonding polymer 40. The binding polymer 40 and the hydrophobic polymer 50 form a hydrophobic layer on the surface of the capped nanoparticle 10. The functionalisable hydrophilic polymer 60 is attached to the hydrophobic polymer 50.

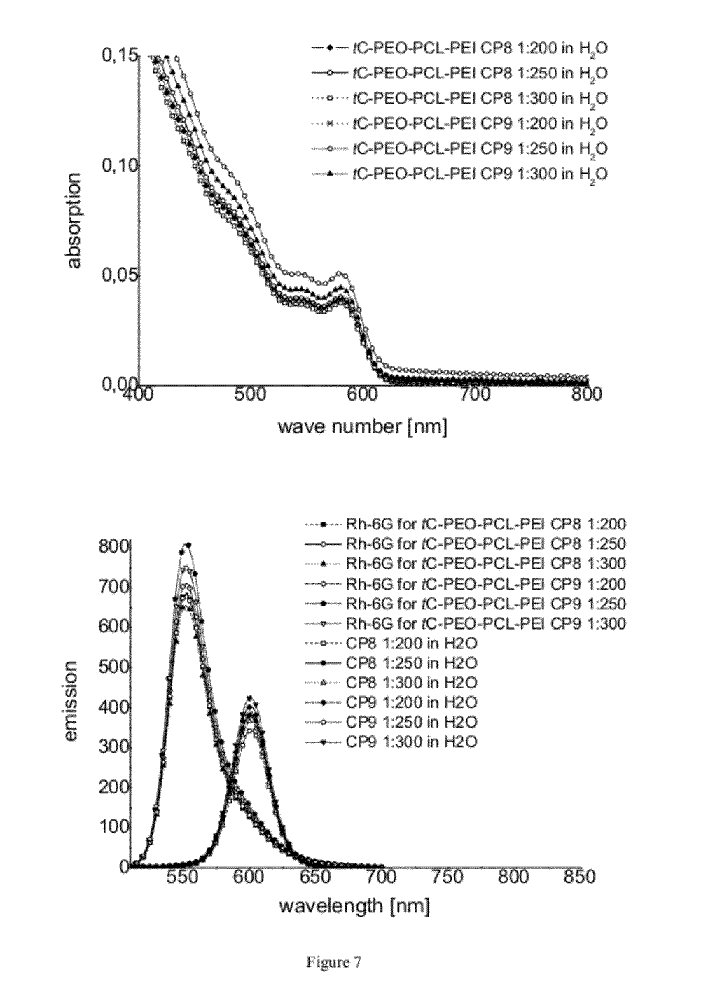

FIG. 7 shows a) absorbance spectra and b) emission spectra of the multi-shell nanoparticle complex 80 of the CdSe/CdS/ZnS (capped nanoparticle 10) with the triblock polymer ligand 30 of tC-PEO-PCL-PEI in water. FIG. 7 shows the effects of highly fluorescent nanoparticle complexes 80 in an aqueous medium. The exiton peak in the UV-vis spectroscopy analysis was observed at 580 nm and the emission maximum at 594 nm.

The dynamic quenching of CdSe/ZnS core/shell nanoparticles, in the presence of super-paramagnetic α-Fe₂O₃, has been debated in the literature (Y. Liu, M. Kim, Y. Wang, Y. A. Wang, X. Peng, Langmuir 2006, 22, 6341). The capped nanoparticles 10 which have the CdSe/ZnS core/shell have been examined in case of core/shell/shell CdSe/CdS/ZnS capped nanoparticles 10 in an example of the present invention. The CdSe/CdS/ZnS capped nanoparticles 10 were packed together with the super-paramagnetic α-Fe₂O₃. The super-paramagnetic α-Fe₂O₃ is packed inside the micellar structure of the nanoparticle complex 80. The packing demonstrates the advantages of the triblock polymer ligand 30. The advantage is that the quenching of the luminescence has been reduced with the super-paramagnetic α-Fe₂O₃ particles.

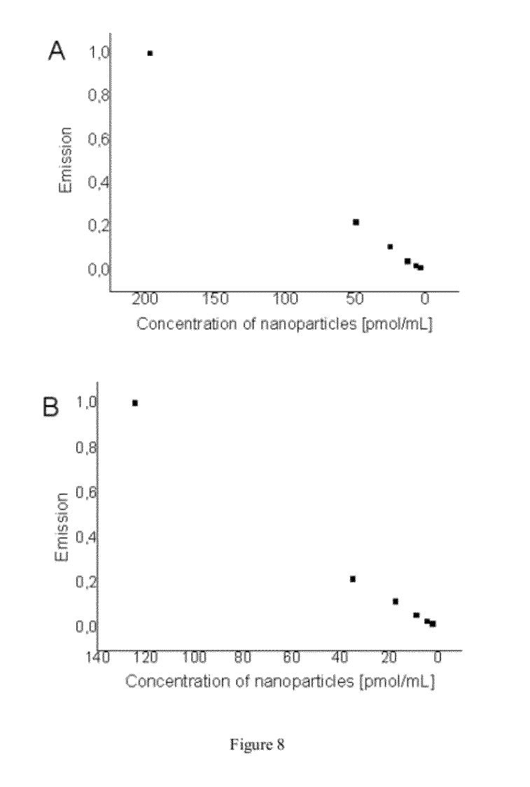

FIG. 8 shows graphs in which the fluorescence intensity of CdSe/CdS/ZnS multi-shell capped semiconducting nanoparticle complex 80 with the ligand tC-PEO-PCL-PEI in an aqueous medium (water) is plotted against the concentration in water. It is observed that due to the PCL hydrophobic polymer 50 the nanoparticle complex 80 shows a steady quantum yield in water during the progress of dilution. Dilution of a 124 pmol/mL aqueous solution of the ligand tC-PEO-PCL-PEI down to 2 pmol/mL (FIG. 8 A) and a 197 pmol to 3 pmol (FIG. 8 B) shows no decrease in the quantum yield of the nanoparticle complex 80. Two different ligand additions were performed and each absolute quantum yield of the highest concentrated probe was equated with one. The results are identical. The capped nanoparticle 10 stays at its lowest attenuation in solution. The low concentration produced a totally clear solution. When using UV-light the uniformly distributed fluorescence in the cuvette could be readily observed. This uniformly distributed fluorescence indicates that very little of the triblock polymer ligand 30 had been removed from the surface of the capped nanoparticle 10. The triblock polymer ligand 30 is therefore responsible for solvating the capped nanoparticle 10 and the nanoparticle complex 80 in the aqueous medium.

The dilution was arranged in 10 mL volumetric flasks. The inspections of the cuvettes with pure water displayed no emission at all. Many research groups have found that the cytotoxicity of semiconducting nanoparticles is dose dependent. Higher concentrations of semiconducting nanoparticles result in a significantly higher death of cells in biological applications (see for example A. M. Derfus, W. C. W. Chan, S. N. Bhatia, Nano Letters 2004, 4, 11; C. Kirchner, T. Liedl, S. Kudera, T. Pellegrino, A. M. Javier, H. E. Gaub, S. Stoelzle, N. Fertig, W. J. Parak, Nano Letters 2005, 5, 331 and A. Shiohara, A. Hoshino, K.-i. Hanaki, K. Suzuki, K. Yamamoto, Microbiology and Immunology 2004, 48, 669).

The observed minimal increase of the quantum yield of the nanoparticle complex 80 is ascribed to the decline of the re-absorption by capped nanoparticle 10 that are in the vicinity of the nanoparticle complex 80. This finding is in agreement with the dilution experiments. Furthermore the monitoring that the quantum yield declines simultaneously with the concentration of the nanoparticle complex 80 is in agreement that the quantum yield stays constant with a dilution of 1:2, the quantum yield of the nanoparticle complex 80 remains constant. For example where the dilution is 50%, then the corresponding quantum yield is 50%. This constant yield of quantum yield testifies that even in high dilutions the triblock polymer ligand 30 remains on the anorganic passivation layer 13 of the capped nanoparticle 10. This finding can be ascribed to the hydrophobic PCL polymer 50.

The drop in the quantum yield can be suppressed by encapsulation of the capped nanoparticle 10 in the micellar structure of the nanoparticle complex 80. It is noted that the preservation of the quantum yield is mainly due to the outer shell 12 of the core/shell/shell capped nanoparticle 10. Where CdS is the intermediate layer of the core/shell/shell. It shows with respect to CdSe a lattice mismatch of approximately 3.6%, thus improving the growths of ZnS shell layer, which has a lattice mismatch of approximately 12% to CdSe. The CdS crystal structure fits the CdSe crystal structure better than the ZnS crystal structure.

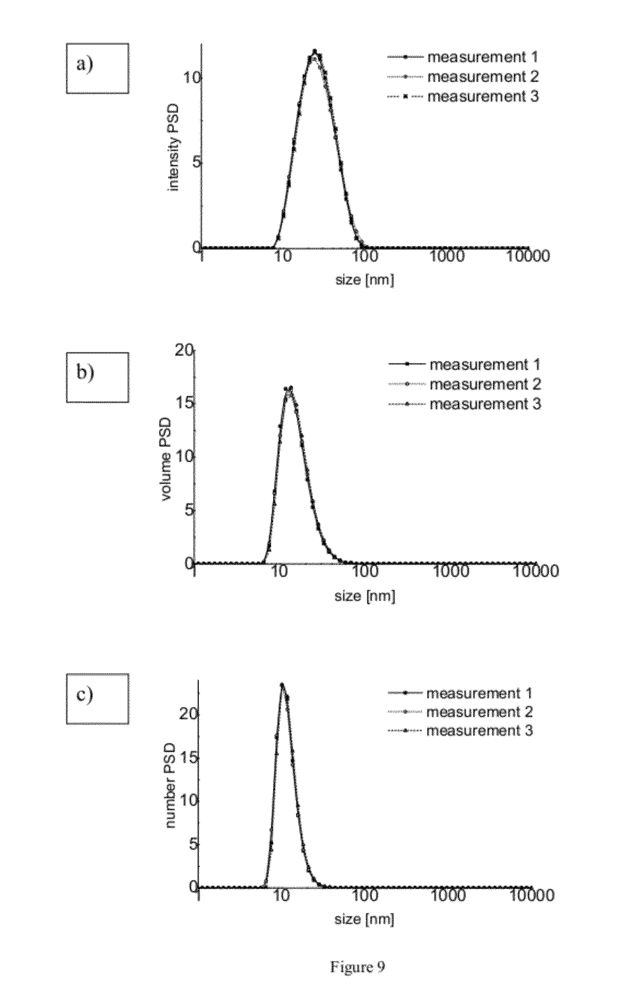

FIG. 9 shows dynamic light scattering of CdSe/CdS/ZnS coated by the ligand DP-PEO-PCL-PEI (DPA) in water, a) DLS intensity b) DLS volume and c) DLS number. The dynamic light scattering (DLS) measurements show a hydrodynamic radius of 27 nm of the nanoparticle complex 80, while the number application c) shows a 16 nm radius of the nanoparticle complex 80. The size range of 16-27 nm is feasible for the nanoparticle complex 80, with respect to the likely expansion of the triblock polymer ligand 30 in solution and an additional solvate shell formed from the solution around the nanoparticle complex 80.

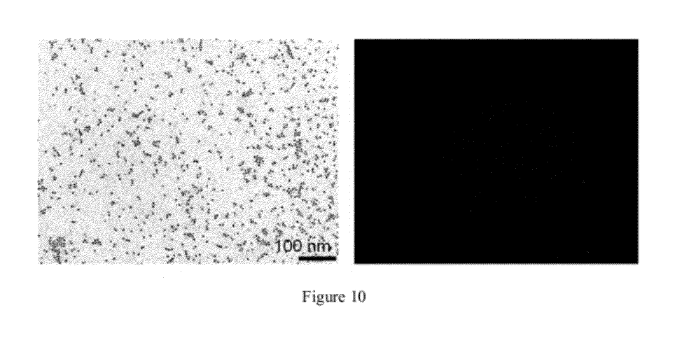

Furthermore, TEM and confocal microscope images, corroborate the presumption of single nanoparticle complexes 80 which are stabilised in an aqueous medium as shown in the schematic of FIG. 10. The confocal microscope images show a uniform luminescence. The uniform luminescence demonstrates that no aggregates have been formed within the nanoparticle complex 80.

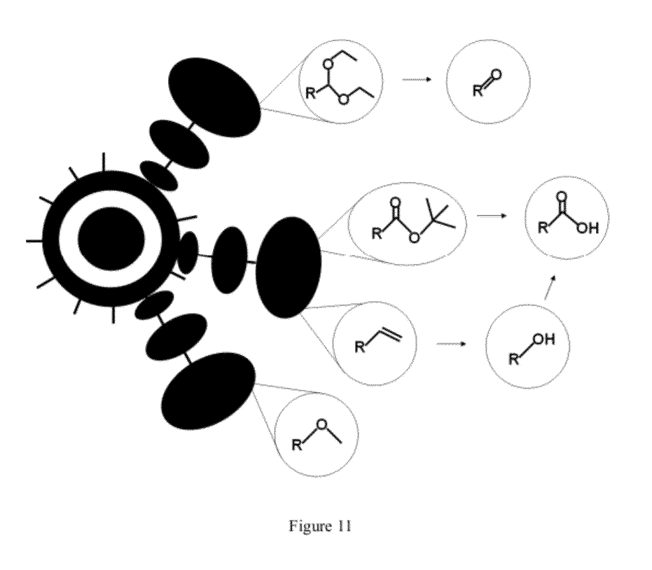

As described the hydrophilic functionalisable polymer 60 has functionalisable terminal groups and by selection of such specific functional terminal groups a variety of different functional groups can be incorporated into the micelles of the nanoparticle complex 80. Due to the functionalisable polymer 60 further functionalisation of the nanoparticle complex 80 can be achieved. Therefore in various aspects of the present invention the hydrophilic functionalisable polymer 60 was provided with terminal functional groups. These terminal functional groups include, but are not limited to, acetyls (DP-), tertiary-butyl protected carboxyl groups (tC-) and methoxy groups (M-). The terminal functional groups can be easily converted into different functional groups as depicted by the illustration in FIG. 11.

The triblock polymer ligand 30 is highly suitable for use in biomedical applications when the triblock polymer ligand 30 has carboxyl terminated functional groups at the functionalisable hydrophilic polymer 60. This is because the carboxyl terminated functional group of the functionalisable hydrophilic polymer 60 can be further functionalised by well known reaction means. Such reaction means include EDC/SulfoNHS-activation of the carboxyl terminated functional group. The EDC/SulfoNHS-activation provides the advantage to facilitate protein coupling to the carboxyl group. In an example of the present invention where the carboxyl terminated functional group of the hydrophilic functionalisable polymer 60 is a tertiary-butyl group, this group can be cleaved by the use of an esterase enzyme.

In terms of the protein coupling of the carboxyl group it is favourable to delimit the amount of terminal functional groups present on each triblock polymer ligand 30 to avoid multi-binding to the same protein (i.e. adjacent triblock polymer ligands 30). Such multi-binding would lead to a loss of flexibility of the micellar structure of the nanoparticle complex 80. The loss of flexibility would also lead to a loss of functionality of the nanoparticle complex 80 used in biological applications. This loss of functionality of the nanoparticle complex 80 would have the same effect of denaturing a protein. This denaturing effect arises since the function of proteins is determined by dimensionally specific parameters of the protein. Therefore in a further aspect of the present invention methoxy-terminated (M-) should preferably be used as the hydrophilic functionalisable polymer 60 segment. The preferable use of methoxy-terminated (M-) hydrophilic functionalisable polymer 60 is because the methoxy-terminated (M-) groups control the number of functional groups on the surface of the micellar nanoparticle complex 80.

In further aspect of the present invention a vinyl and allyl terminated polyethylene oxide (PEO) as the hydrophilic functionalisable polymer 60, purchased from Clariant Functional Chemicals, was combined with the PCL (hydrophobic polymer 50) and the PEI (binding polymer 40) to manufacture the triblock polymer ligand 30. The double bond present in the allyl and the vinyl groups can be verified by ¹H NMR spectroscopy in the region of 6.6 ppm. The double bond opens the opportunity to use the triblock polymer ligand in the field of click chemistry, cycloaddition as well as transition metal catalysed reactions as coupling reactions. The electrophilic bromination of the terminal double bond, permits Suzuki coupling using catecholborane, is a selective reaction, where other functional groups of the triblock polymer ligand 30 are not affected. Supplemental acidic treatment of the double bond in an aqueous solution generates a terminal hydroxyl group, whilst a urethane group of the resultant triblock polymer ligand 30 is buried in a protecting, hydrophobic environment. This terminal function allows inter alia the reaction with a succinic acid moiety to obtain a carboxyl group as well as the coupling via CDI or epichlorohydrin.

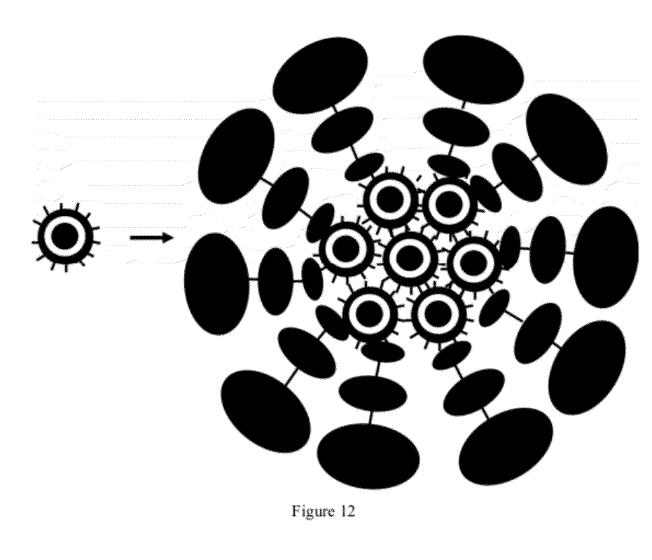

A further advantage of the triblock polymer ligand 30 according to the present invention is that independent of the ratio of the capped nanoparticles 10 to triblock polymer ligand 30, the nanoparticle complex 80 will have a micellar-like structure. The micellar structure of the nanoparticle complex 80 can contain one or more capped nanoparticle 10 (as shown in the schematic of FIG. 12).

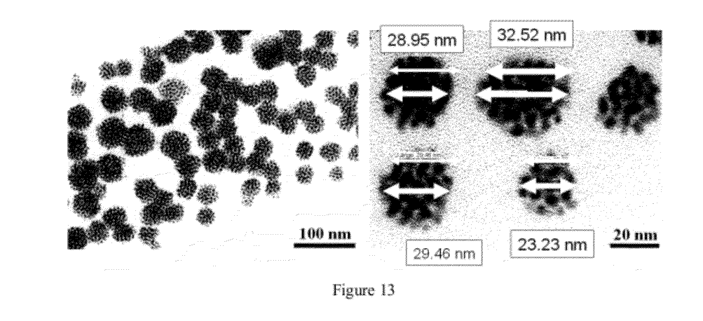

With reference to FIG. 13, this shows the TEM images of core/shell/shell nanoparticle complexes 80 in water. FIG. 13 exemplifies that even under high vacuum conditions required for obtaining TEM images the three dimensional micellar structure of the nanoparticle complex 80 is maintained. The micellar structure of the nanoparticle complex 80 has an inner diameter of approximately 31 nm in this example.

It is observed that the higher the ratio of triblock polymer ligand 30 to capped nanoparticle 10, the more of the triblock polymer ligand 30 is available per capped nanoparticle 10. The higher the total surface area of the capped nanoparticle 10, the more likely it is that a single capped nanoparticle 10 will be at the centre of the nanoparticle complex 80.

Furthermore by decreasing the ratio of capped nanoparticle 10 to triblock polymer ligand 30 to a threshold amount it is observed that insufficient triblock polymer ligand 30 is available to stabilise the surface of the capped nanoparticle 10. Accordingly the capped nanoparticles 10 form an aggregate to minimise the surface of the capped nanoparticle 10, towards the solvent. Such new aggregate formations can be encouraged by the manipulation of the triblock polymer ligand 30 through the hyper branched binding that is provided by the binding polymer 40. In this aspect of the present invention several capped nanoparticles 10 can be within the centre of the micellar structure of the nanoparticle complex 80, as shown in FIG. 12.

It has been reported that clustering of super-paramagnetic magnetite nanoparticles results in a higher saturation of magnetisation than that of individual magnetite nanoparticles (Ge, J., Y. Hu, et al. (2007). “Superparamagnetic Magnetite Colloidal Nanocrystal Clusters.” Angewandte Chemie International Edition 46 (23): 4342-4345). This saturation of magnetisation correlates with the cluster size or the number of particles inside the micelle, respectively.

The controlled clustering which is possible simply by tuning the ratio of triblock copolymer 30 to capped nanoparticle 10 allows the controlled clustering of iron oxide particles.

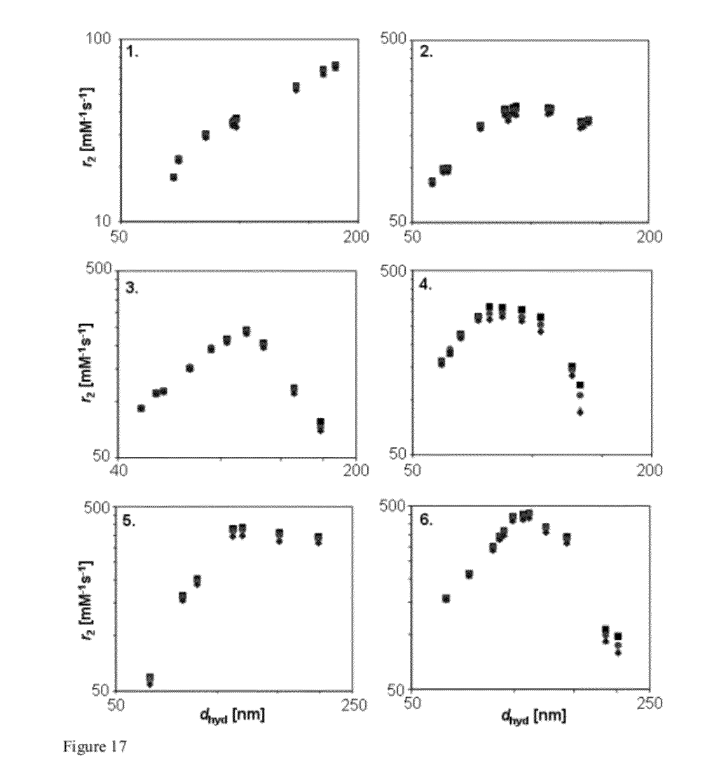

Such iron oxide particles are highly interesting as contrast agents in magnetic resoncance imaging. By controlling the size of the cluster the r2 relaxivity can be adjusted to its maximum value, which is the static dephasing regime (FIG. 17, 2-6). In the motional average regime and the echo limiting regime lower relaxivities are observed. The feasibility to control the cluster size and thus the relaxity makes these clusters highly suitable as a negative contrast agent in MRI.

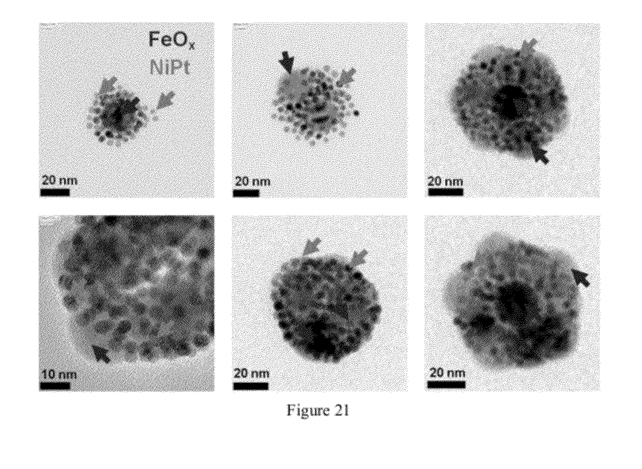

The clustering is not only possible for one species of nanoparticles like the ones mentioned above [0053] but also a number of nanoparticles made from different materials can be encapsulated in one micelle. As an example superparamagnetic iron oxide nanoparticles and NiPt nanoparticles were coencapsulated with triblock copolymer ligand 30 (PEO-PCL-PEI) as can be seen in FIG. 21.

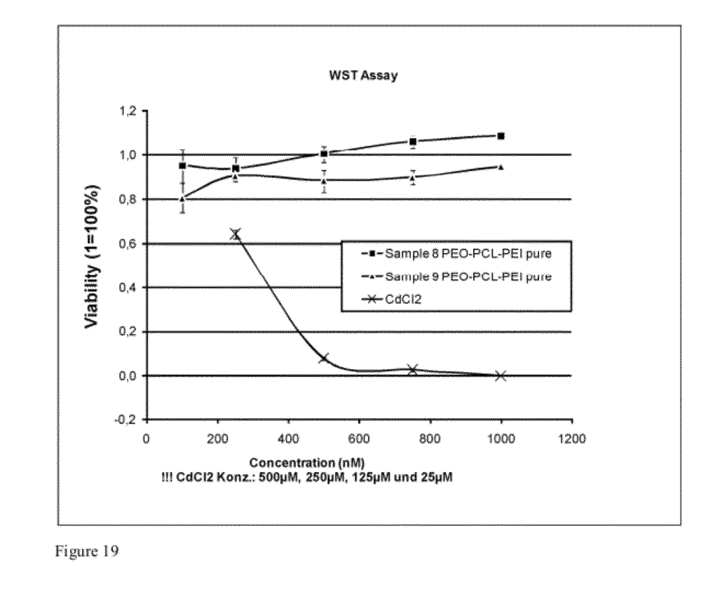

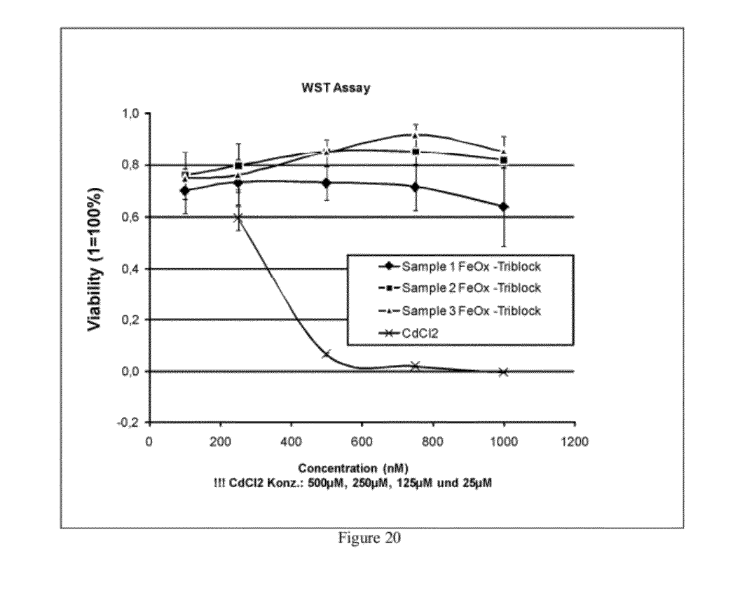

The cytotoxiciy has of the pure triblock copolymer PEO-PCL-PEI has been assessed in a WST-assay shown in FIG. 19. No concentration dependent effect on cell viability can be observed. In the same context the cytotoxicity of superparamagnetic iron oxide nanoparticle clusters inside the PEO-PCL-PEI triblock copolymer complex have been measured (FIG. 20). Also the WST-assay of the iron oxide nanoparticles inside the nanoparticle complex 80 does not show any concentration dependent effect on cell viability which might lead to cytotoxicity.

During the synthesis of the nanoparticle complex 80, it is known that the rate of water addition to the mixture 65, in which the capped nanoparticles 10 and the triblock polymers 30 are present, and when phase transfer occurs, the phase transfer provides a means to control the size of the micellar structures of the nanoparticle complex 80. See Schabas et al (G. Schabas, H. Yusuf, M. G. Moffitt, D. Sinton, Langmuir 2008, 24, 637). Schabas et al describe that the faster water is added to the mixture 65 then the faster the manufacture of the nanoparticle complex 80 will occur. This increased rate of manufacture of the nanoparticle complex 80 results in a corresponding decrease in the mean particle diameter of the nanoparticle complex 80.

The versatile options of the unique triblock polymer ligand 30 have been also achieved by manufacturing the nanoparticle complexes 80, where the capped nanoparticle 10 is InP, NaYF₄, PbS, Au, FePt, iron oxide and NiPt. These capped nanoparticles 10 are not always surrounded by a TOP/TOPO passivation layer. Therefore the manufacture of nanoparticle complexes 80 works with many hydrophobic capped nanoparticles and is not limited to semiconducting nanoparticles.

Single as well as multi nanoparticle complexes 80 of micellar structure can be formed by varying the capped nanoparticle 10 to triblock polymer ligand 30 ratios. It is concluded that the triblock polymer ligand 30 does not make demands in terms of the amount of capped nanoparticle 10 present. In the ambit of semiconducting capped nanoparticles 10 the triblock polymer ligand 30 has no difficulties with different shell materials of the cores of the capped nanoparticles 10.

Therefore, different capped nanoparticles 10 such as CdSe, as well as CdSe/CdS or CdSe/CdS/ZnS core, core/shell and core/shell/shell nanoparticles have been solubilised in an aqueous medium with ease and quantitatively in examples of the present invention. Therefore the triblock polymer ligand 30 provides the opportunity to stabilise the quantum yield by the organic passivation layer provided by the triblock polymer ligand 30. This fact is a benefit regarding essential buffer tests. The inorganic shell stabilises the quantum yield of the nanoparticle complex 80.

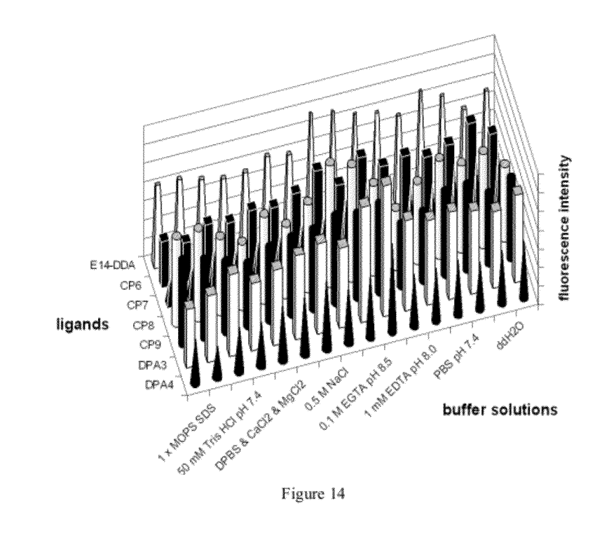

To verify the impact of buffer solutions on exciton relaxation, the same amount of capped nanoparticle 10 was added to a 200 fold excess of triblock polymer ligand 30 and transferred into water. The solution was purified by use of a 0.2 μm PTFE-filter, from this stock solution the same amount of nanoparticle complex 80 was always re-dissolved in a different buffer solution. The incubation was performed for two hours. With reference to FIG. 14 the integral of the area of the emission peaks is compared for different triblock polymer ligands 30. By comparison of the quantum yield in each alignment to the quantum yield of the capped nanoparticles 10 in water, it appears that the quantum yield of the nanoparticle complex 80 never drops by more than 7%.

FIG. 14 shows the repeated determination of the area of the emission peaks of multi-shell CdSe/CdS/ZnS capped nanoparticles 10 coated by DP-PEO-PCL-PEI, tC-PEO-PCL-PEI and M-PEO-PEI-DDA in water, 1×MOPS SDS, 50 mM Tris HCl pH 7.4, DPBS & CaCl₂ & MgCl₂, 0.5 M NaCl, 0.1 M EGTA pH 8.5, 0.1 M EDTA pH 8.5, PBS pH 7.4.

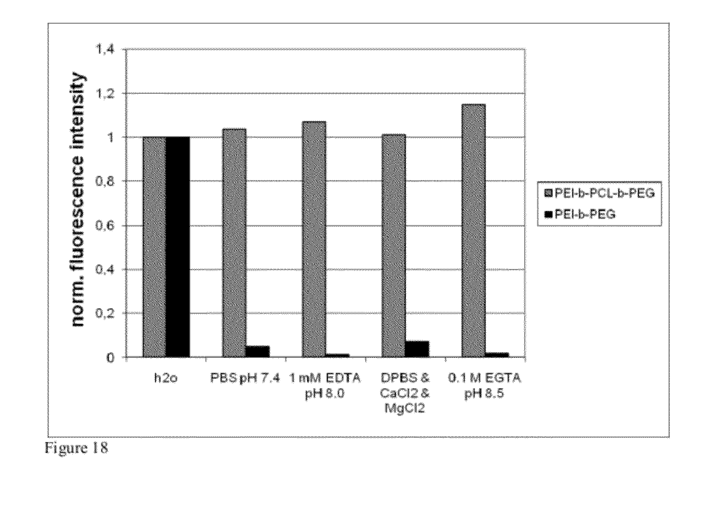

Apart from stability tests of the triblock copolymer-nanoparticle complexes in various buffer solutions a comparison was made to evaluate the effect of the additional hydrophobic polymer block 50. Therefore the fluorescence intensity of semiconductor quantum dot polymer complexes made from a triblock copolymer b-PEO-b-PCL-b-PEI and a diblock copolymer b-PEO-b-PEI were measured in pure water and different buffer solutions as can be seen in FIG. 18.

In all cases it can be seen from FIG. 18 that the introduction of a hydrophobic segment into the polymer ligand leads to increased stability against aqueous solutions with high ionic strength. This demonstrates the effective shielding of the incorporated nanoparticles from the environment through the additional hydrophobic polymer block 50.

Prospects are published using PEO-phospholipid micelles, which contain different kind of nanoparticles, whereby a combination of properties is achieved. In the case of quantum dots with their optical and magnetite nanoparticles with their magnetic properties a nanocomposite is created, which is simultaneous and detected via fluorescence spectroscopy and magnetic resonance imaging (MRI). Those multifunctional nanoparticles open up a wide field of application in the biomedical imaging in vivo and in vitro, even having the potential to integrate therapeutic and diagnostic functions into a single nanodevice. Previous in vitro studies have demonstrated that drug molecules and magnetic particles can be incorporated within a micelle to enable the corroboration of drug delivery by MRI.

Using triblockpolymers and coupling strategies not only the encapsulation in one micelle but also in certain distances on the surface is feasible. Those multifunctional nanocomposites should overcome due to the hydrophobic packing the obstacle of other hybrid nanosystems in vivo studies, in particular for cancer imaging and therapy, owing to low stability or short systemic circulation times.

In the domain of nano-architecture among the construction of superior structures all the way to meta-structures the multifunctional nanoparticles complexes 80 can be equipped with further markers like radioactive compounds (¹⁸F), conducting molecules, catalysts, and drugs.

The triblock polymer ligand 30 stabilises the luminescence of the capped nanoparticles 10 (see FIG. 14) and the triblock polymer ligand 30 provides functional groups for further coupling reactions.

The hydrophobic polymer 50 (PCL segment) shields the surface of the capped nanoparticle 10 from the aqueous medium. This shielding of the surface of the capped nanoparticle 10 is reflected in the minimisation of the loss in fluorescence of capped nanoparticles 10 when used in biological applications. The potential to use the capped nanoparticles 10 of CdSe/CdS/ZnS with bulkier ZnS shells provides a means to use nanoparticle complexes 80 in biological applications.

Nanoparticle complexes 80 were also manufactured with nanoparticles other than CdSe based semiconductore nanoparticles such as InP, NaYF₄, GdPO₄, PbS, Au, FePt, iron oxide and NiPt. Therefore many hydrophobic nanoparticles can form the nanoparticle complex 80 with the triblock polymer ligand 30. In the same way as with the semiconducting nanoparticles the non semi-conducting nanoparticles are functionalisable nanoparticle complexes 80 with the triblock polymer ligand 30. It has been observed that the manufacture of the nanoparticle complex 80 is not restricted by the shape of the capped nanoparticle 10.

The phase transfer of the nanoparticle complex 80 from an organic solvent to an aqueous medium is therefore also possible with capped nanoparticles 10 that are in the shape of rods, dots, tetrapods and tubes.

Since the nanoparticle complex 80 has terminal functional groups at the functionalisable hydrophilic polymer 60 on the triblock polymer ligand 30, coupling reactions can be achieved at the hydrophilic functionalisable polymer 60 with various chemical groups. These chemical groups can be further reacted to molecules like proteins, antibodies, carbohydrates, DNA- & RNA-fragments, drugs. These chemical groups can also be further reacted to larger constructs like other particles or solid surfaces.

The triblock polymer ligand 30 provides new perspectives for applications in magnetic resonance imaging (MRI). This is achieved by the encapsulation of one to 200 particles of capped nanoparticles 10 (e.g. iron oxide alone or together with gold nanoparticles) in the micellar structure of the nanoparticle complex 80.

In further aspect of the present invention mixtures of different types, sizes and shapes of the capped nanoparticles 10 can be used to manufacture the nanoparticle complex 80. The mixtures of capped nanoparticles 10 can be encapsulated in the same micellar structure of the nanoparticle complex 80. This encapsulation of mixtures of capped nanoparticle 10 increases the prospects of using the resultant manufactured nanoparticle complex 80 in fluorescent labelling applications. It is envisaged that magnetic nanoparticles and semiconducting nanoparticles can be encapsulated in the centre of the nanoparticle complex 80. The magnetic nanoparticles and semiconducting nanoparticles could therefore provide a twofold detection means of the resultant nanoparticle complex 80. The twofold detection means is that the magnetic nanoparticles can be detected by magnetism and the semiconducting nanoparticles can be labelled and detected by florescent tracing. This twofold detection means is advantageous when the nanoparticle complex 80 is used in biological applications.

The different biomolecules used to further functionalise the nanoparticle complex 80 can be tuned with additional markers such as dyes or radioactive markers. The marking of the different biomolecules opens the pathway to use the nanoparticle complex 80 in biological applications. The micellar structure of the nanoparticle complex 80 which is functionalised with an antibody can be traced using FRET measurements.

In a further example of the invention it is envisaged that magnetically capped nanoparticles 10 can be used widely in industrial applications. Magnetically capped nanoparticles 10 will interact with a magnetic field. This interaction of a magnetic nanoparticle complex 80 with the magnetic field can be used to direct the magnetic nanoparticle complex 80 to a specific target using the magnetic field. A magnetic nanoparticle complex 80 can also be used in biomedical applications e.g. as a contrast agent in computer tomography or magnetic resonance imaging. It is envisaged that one application of the magnetic nanoparticle complex 80 is in drug-delivery systems. The magnetic nanoparticle complex 80 can be directed to a biological target by the interaction with the magnetic field. Furthermore magnetic isolation of a magnetic nanoparticle complex 80 can be used in antibody selection.

Due to the naturally uniform micellar structure of the nanoparticle complex 80, the nanoparticle complex 80 can be used to coat a surface of an object with the capped nanoparticle 10. The use of the nanoparticle complex 80 can be used to provide a fine coverage on the surface with the capped nanoparticle 10. In such applications where the nanoparticle complex 80 is used to coat the surface, the triblock polymer ligand 30 can be removed from the capped nanoparticle 10 by chemical means or with a laser thus providing a uniform deposition of the capped nanoparticle 10 on the surface. In a further example of the invention for coating a surface, the deposition density of the capped nanoparticle 10 on the surface can be tuned by using nanoparticle complexes 80 with a different length of triblock polymer ligand 30. The length of the triblock polymer ligand 30 will therefore determine the deposition density of the capped nanoparticle 10 on the surface. In a further example of the invention the surface to be coated by the nanoparticle complex 80 can be made hydrophilic by the deposition of the nanoparticle complex 80 on the surface. Conversely in a further example the surface can be made hydrophobic (reminiscent of the lotus effect) by removal of the triblock polymer ligand 30 from the nanoparticle complex 80. In a further aspect the modification of the surface can also achieve opalescence when coated with the nanoparticle complex 80.

### Examples

### Preparation of Capped Nanoparticles 10

TOP/TOPO capped CdSe/CdS/ZnS core-shell-shell nanoparticles 10, were synthesised by methods reported previously (I. Mekis, D. V. Talapin, A. Kornowski, M. Haase, H. Weller, Journal of Physical Chemistry B 2003, 107, 7454 and D. V. Talapin, A. L. Rogach, A. Kornowski, M. Haase, H. Weller, Nano Letters 2001, 1, 207. The capped nanoparticles 10 were precipitated twice with methanol to remove excess amounts of TOP/TOPO and were stored in anhydrous chloroform as a solution.

### Polymer Synthesis

The synthesis of the diblock polymer 20 PEO-PCL was achieved catalytically (see FIG. 1 and the associated discussion).

### Synthesis of Triblock Polymer 30

### Synthesis of the Hydrophilic Polymer 60

Under an atmosphere of argon, 2.1 g naphthalene (16.0 mmol) and 567.0 mg potassium (14.5 mmol) were dissolved in 20 mL anhydrous THF and stirred over night. A PEO initiator (16.0 mmol) was added and the reaction stirred for 48 hours.

Three different reactions were performed using three different initiators for the anionic ring opening polymerisation: tert-butyl-3-hydroxypropionate (tC-), 3,3-dieethoxy-propanol (DP-), 2-methoxyethanol (M-).

In the three different reactions, to 500 mL anhydrous THF and 16 mL ethylene oxide in a 1 L flask 14.5 mmol of the initiator was injected and the reaction was stirred at 40° C. for three days.

The purification of three derivatives of the poly (ethylene oxide) (PEO) hydrophilic polymer 60 was achieved by precipitation of the hydrophilic polymer 60 using an excess of diethyl ether. Analysis involved ¹H- and ¹³C-NMR, HMBC, HHCosy, HSQC, TOCSY, Maldi-TOF, GPC, FT-IR and TG measurements. (See FIG. 3) ¹H-NMR of M-PEO-PCL-PH (upper scheme), DP-PEO-PCL-PH (middle scheme), tC-PEO-PCL:-PEI (lower scheme),

### Synthesis of PEO-b-PCL Diblock Polymer 20

The hydrophobic block 50 was synthesised by ring-opening polymerisation of ε-caprolactone using the three derivatives of the poly (ethylene oxide) (PEO) hydrophilic polymer 60 as initiator as well as purchased PEO polymers like Allyl-PEO and Vinyl-PEO (Clariant Functional Chemicals) and followed the procedure as disclosed by R. F. Storey, J. W. Sherman, Macromolecules 2002, 35, 1504. The dried derivatives of the poly (ethylene oxide) (PEO) hydrophilic polymer 60 was weighed into a flask and ε-caprolactone (different amounts according to different M/I ratios) was subsequently added. The reaction mixture was heated up to 130° C. before one drop of stannous octanoate catalyst was added. The polymerisation was performed for 3 hours.

The resulting viscous solution was rapidly cooled, upon which it solidified. The crude polymer was dissolved in dichloromethane and precipitated twice by the addition of heptane. Afterwards the product was analysed by ¹H- and ¹³C-NMR, HMBC, HHCosy, HSQC, TOCSY, GPC, FT-IR and TG measurements.

### Triblock Polymer Ligand 30 Synthesis

The coupling to the amines was achieved by activation with 1,1′-carbonyldiimidazol (CDI). The diblock polymer ligand 20 was coupled to poly (ethylene imine) by activation of the hydroxyl group of the PEO-PCL derivatives by a fifteen fold excess of 1,1-carbonyldiimidazol (CDI) in dry chloroform. After 2 hours of stirring at room temperature, the excess CDI was hydrolysed by a twofold extraction each with 5 mL water and drying over sodium sulfate. The solid phase was filtered off and poly(ethylene imine) with a mass of ˜700 g/mol (PEO-PCL to PEI ratio 1-3 to 1) was added to each reaction mixture.

The reaction mixture was stirred for 5 hours at a temperature of 55° C. The product was precipitated by the addition of diethyl ether and dialysed to remove the imidazole side product, which would prejudicial interfere with the nanoparticle surface. The triblock polymers 30 were analysed by ¹H- and ¹³C-NMR, HMBC, HHCosy, HSQC, TOCSY, GPC, FT-IR and TG measurements. See FIG. 4.

### Synthesis of M-PEO-N4

A 1.3 fold excess of CDI activated poly (ethylene oxide) mono methyl ether (M=1100 g/mol, M-PEO₁₁₀₀, Sigma Aldrich) was reacted with tris (2-aminoethyl) amine in anhydrous chloroform for 12 hours at 55° C. The product was precipitated three times in diethyl ether. See FIG. 5, ¹H-NMR of M-PEO-N4.

### Complexation of the Capped Nanoparticle 10 with Triblock Polymer 30 to Manufacture Nanoparticle Complex 80

The concentration of the capped nanoparticle 10 in an anhydrous chloroform solution was determined by UV-Vis absorbance spectroscopy. A certain amount of the capped nanoparticles 10, usual amounts range from 0.5 to 10 nmol, was degassed and dehumidified under a nitrogen flow and re-dissolved in 100 μL of tetrahydrofuran to form a solution.

A 250 molar excess of the triblock polymer ligand 30 was dissolved in anhydrous THF and incubated with the capped nanoparticles 10.

The incubation time was 10 minutes. After the edition of approximately 400 μL of water the THF was evaporated first under a nitrogen atmosphere and then under reduced pressure.

A purification step by filtering over a 0.2 μm syringe filter is used to isolate the nanoparticle complex 80.

### Hydrolysis of tC-PEO-PCL-PEI

Porcine liver esterase (Fluka 46058, ≧130 units/mg) was added to a solution of the triblock polymer ligand 30 tC-PEO-PCL-PEI (0.64 mmol) in 0.1 M potassium phosphate (pH 7.5, 10 mL) containing 0.5 mL methanol, the mixture was stirred at room temperature overnight.

The solvent was removed in vacuum before the resulting residue was dissolved in chloroform and the desired product was precipitated by the addition of cold diethyl ether (8° C.) and collected by filtration.

Furthermore it is envisaged that the following further examples can be implemented using the teachings of the present invention.

Alternatives for the hydrophilic functionalisable polymer 60 (PEO), would be polypropyleneoxide and.

Alternatives for hydrophobic polymer 50 are polystyrene, polylactide, polyester, polyglycolids, polyisoprene, polyethylene, polypropylene and polyamide.

Alternatives for the binding motif 40 are polymers which contain nucleophilic groups or hydrophobic side chains. Furthermore non polymeric binding motifs 40 such as oligocystein, dihydrolipoic acid or low molecular weight amines can be used.

Having thus described the present invention in detail, it is to be understood that the foregoing detailed description of the invention is not intended to limit the scope of the invention thereof. The person skilled in the art will recognise that the invention can be practiced with modification within the scope of the attached claims. At least, it should be noted that the invention is not limited to the detailed description of the invention and/or of the examples of the invention. What is desired to be protected by letters patent is set forth in the following claims.

### REFERENCE NUMERALS

10—capped Nanoparticle

11—core

12—Shell

20—diblock polymer

30—triblock polymer ligand

40—Binding motif

50—Hydrophobic polymer

60—Hydrophilic functionalisable polymer

65—mixture

70—liquid

80—Nanoparticle complex

1. A nanoparticle complex comprising a plurality of triblock polymer ligands attached to a capped nanoparticle, wherein the triblock polymer ligand comprises
a. a functionalisable hydrophilic polymer, which promotes solubility of the nanoparticle complex in water or water miscible organic solvents like THF respectively
b. a hydrophobic polymer or oligomer,
c. a hydrophilic or hydrophobic binding polymer or oligomer, comprising groups with an affinity to the nanoparticle surface.

2. The nanoparticle complex according to claim 1, wherein the hydrophilic functionalisable polymer is poly (ethylene oxide) (PEO), polyalcohols; polysugars like dextranes; polyoxazolines, hydrophilic (poly-)peptides.

3. The nanoparticle complex according to claim 1, wherein the hydrophobic polymer or oligomer is selected from the group of Polyesters like Polyethylene terephthalate (PET), Polyethylene naphthalate (PEN), Polybutylene terephthalate (PBT), Polytrimethylene terephthalate (PTT), Polyglycolide or Polyglycolic acid (PGA), Polylactic acid (PLA), Polycaprolactone (PCL), Polyethylene adipate (PEA), Polyhydroxyalkanoate (PHA), Vectran, Polycyclohexylenedimethylene terephthalate (PCT); Polycarbonates like allyl diglycol carbonate (ADC), hydrophobic peptides like transmembrane helices from transmembrane proteins; Polyethers; Polyetherimides; Polyesteramides; Polyamides like Polycaprolactam, Acrylate Polymers like Poly(methyl methacrylate) (PMMA); Polyvinyl acetate (PVAc)Polymethacrylmethylimid (PMMI); Polyamide-imides; Polyether ether ketone (PEEK); Polyimide like Polybismaleinimid (PBMI); Polyureas; Polyvinylpyrrolidon (PVP) or Polyhydantoine

4. The nanoparticle complex according to claim 1, wherein the hydrophobic polymer is poly (ε-caprolactone) (PCL).

5. The nanoparticle complex according to claim 1, wherein the binding polymer or oligomer exhibits groups like amines, thiols, phosphines, carboxylates, dithiocarbamates or carbodithioate as affinity groups to the nanoparticle surface.

6. The nanoparticle complex according to claim 1, wherein the binding motif is poly (ethylene imine) (PEI) and/or comprises a plurality of primary and/or secondary amines, preferably between 6 to 8 primary/secondary amines.

7. The nanoparticle complex according to claim 1, wherein the triblock polymer is attached to the nanoparticle via the affinity groups of the binding polymer.

8. The nanoparticle complex according to claim 1, wherein the hydrophobic polymer or oligomer forms a hydrophobic corona around the capped nanoparticle.

9. The nanoparticle complex according to claim 1, wherein the triblock polymer ligand is additionally stabilized through the formation of hydrogen bonds between the binding motif and the hydrophobic polymer, wherein the binding motif and the hydrophobic polymer act both as hydrogen bond donor or acceptor, respectively.

10. The nanoparticle complex according to claim 1, wherein the triblock polymer (30) shows no cytotoxicity in in-vitro assays.

11. The nanoparticle complex according to claim 1, wherein the triblock copolymer alone is able to form micellar structures in water.

12. The nanoparticle complex according to claim 1, wherein the capped nanoparticle is a hydrophobic capped nanoparticle.

13. The nanoparticle complex according to claim 12, wherein the hydrophobic capping ligand of the capped nanoparticle comprises at least one alkyl chain attached to an affinity group selected from the group comprising phosophine, phosphine oxide, amine, thiole, carboxylic acid.

14. The nanoparticle compelx according to claim 1 wherein the triblock copolymer ligand can encapsulate one or more capped nanoparticles of one or more particle species in one nanoparticle complex.

15. The nanoparticle compelx according to claim 1 wherein the number of encapsulated nanoparticles inside the micelle can be adjusted through the ratio of triblock copolymer ligands to capped nanoparticles.

16. The nanoparticle complex according to claim 1, wherein the capped nanoparticle is selected from the group comprising semiconductors materials of
a. the 2A/B group and 6A group of the periodic table like CdS, CdSe, ZnO, ZnS or CdTe or the group comprising elements of the 4A group and 6A group of the periodic table like PbS
b. the 3A group and 5A group of the periodic table like InP or InAs
c. the 1A, 3A and 6A group of the periodic table like CIS or CIGS (Cu(In,Ga)(S,SE)₂

17. The nanoparticle complex according to claim 1, wherein the capped nanoparticle is selected from the group comprising materials of
a. metals like gold, silver or platinum
b. an alloy like NiPt, FePt,
c. a metal oxide like Fe₂O₃ or Fe₃O₄.
d. a rare earth doped nanoparticles like NaYF₄, GdPO₄

18. The nanoparticle complex according to claim 16, wherein the capped semiconductor nanoparticle is a core/shell/nanoparticle and the shell material is selected from the group comprising elements of the 2A/B group and 6A group of the periodic table like CdSe, CdS ZnS or alloys thereof

19. The nanoparticle complex according to claim 16, herein the capped semiconductor nanoparticle is a core/shell/shell nanoparticle and both shell materials and are selected from the group comprising elements of the 2A/B group and 6A group of the periodic table like CdSe, CdS ZnS or alloys thereof.

20. Use of a nanoparticle complex according to claim 1 in biological applications for the attachment to enzymes, florescence and biomedical imaging.

21. Use of the nanoparticle complex according to any of claim 1 as contrast agent in MRT imaging and/or further medical diagnostics or combinations of diagnostic methods.

22. Use of the nanoparticle complex according to claim 20 comprising iron oxide, wherein the saturation magnetization of the clusters can be maximized through adjustment of the cluster size to yield maximum contrast in MRT.
